(19) United States
(12) Patent Application Publication
(10) Pub. No.: US 2023/0277419 A1
(43) Pub. Date: Sep. 7, 2023

(54) MAKE-UP REMOVER WIPE FORMULATIONS

(71) Applicant: Bausch + Lomb Ireland Limited, Dublin (IE)

(72) Inventor: Martin COFFEY, Rochester, NY (US)

(21) Appl. No.: 18/006,917

(22) PCT Filed: Jul. 30, 2021
(86) PCT No.: PCT/IB2021/057006

§ 371 Date: Jan. 26, 2023

Related U.S. Application Data
(60) Provisional application No. 63/059,395, filed on Jul. 31, 2020.

(51) Int. Cl. A61K 8/02; A61K 8/60; A61K 8/97; A61K 8/67; A61K 8/34; A61K 8/362; A61K 8/73; A61K 8/49; A61Q 1/14

(52) U.S. Cl. CPC A61K 8/0208; A61K 8/345; A61K 8/362; A61K 8/4953; A61K 8/604; A61K 8/67; A61K 8/735; A61K 8/97; A61Q 1/14

(57) ABSTRACT

The present disclosure describes pre-moistened wipes and cleansing formulations and methods for cleansing the eyelid, periocular, and ocular surface tissues. The formulations comprise mixtures of gentle surfactants and naturally -derived ingredients, which provides for effective cleaning of delicate skin while having non-irritating properties.

Formulations and methods for cleansing the eyelid, periocular, and ocular surface tissues are provided herein. In some embodiments, disclosed herein are formulations comprising mixtures of gentle surfactants and naturally-derived ingredients.

The present disclosure is related to pre-moistened wipes and cleansing formulations, more particularly lid wipes and cleansing formulations comprising at least one surfactant which provides for effective cleaning of delicate skin while having non-irritating properties, making it useful for periocular and ocular surface tissues.

In some embodiments, there is provided a wipe for the eye area, comprising a pad which is pre-moistened with a cleansing formulation. In some embodiments, the pre-moistened pad is in a package. In some embodiments, the package is a foil-foil package. In some embodiments, the pre-moistened pad is in a sealed package. In some embodiments, the formulation comprises decyl glucoside and/or sorbitan oleate decylglucoside crosspolymer (sugamulse D9). In some embodiments, the formulation further comprises at least one natural humectant. In some embodiments, the formulation further comprises at least one plant extract. In some embodiments, the formulation further comprises at least one buffer. In some embodiments, the formulation further comprises at least one natural humectant, at least one plant extract, and at least one buffer. In some embodiments, the formulation is sterilized by filtration, autoclaving, gamma irradiation and/or electron-beam. In some embodiments, the formulation is compatible with sterilization by filtration, autoclaving, gamma irradiation and/or electron-beam. In some embodiments, the formulation is stable. In some embodiments, the formulation is shelf-stable.

Also disclosed herein are methods for cleansing, cleaning, conditioning, moisturizing, lubricating, soothing, calming, refreshing, whitening, lightening, and/or brightening the eyelid, periocular, and/or ocular surface tissues. Also disclosed herein are methods for treating, improving, or ameliorating inflammation; removing irritants, makeup, debris, crusting, and/or excess oil or secretions; maintaining eyelid hygiene; and/or treating, improving, and/or ameliorating blepharitis and symptoms thereof.

Definitions of certain terms as used in this application are provided below. Unless defined otherwise, all technical and scientific terms used herein have the normal and common meaning that would be commonly understood by one of ordinary skill in the art to which this disclosure belongs.

As used herein, “a,” “an,” and “the” refer to one or more (i.e., to at least one) of the grammatical object of the article.

As used herein, the terms “treat” and “treatment” include medical management of a disease, disorder, and/or condition of a subject as would be understood by a person of ordinary skill in the art. (See, e.g., Stedman’s Medical Dictionary.) In general, an appropriate dose and treatment regimen provide at least one of the formulations of the present disclosure sufficient to provide therapeutic and/or prophylactic benefit. For both therapeutic treatment and prophylactic or preventative measures, therapeutic and/or prophylactic benefits include, for example, an improved clinical outcome, wherein the object is to prevent or slow or lessen an undesired physiological change or disorder, or to prevent or slow or lessen the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer symptom-free status (i.e., decreasing the likelihood or the propensity that a subject will present symptoms on the basis of which a diagnosis of a disease is made); diminishment of extent of disease, disorder, and/or condition; stabilized (i.e., not worsening) state of disease, disorder, and/or condition; delay or slowing of progression of a disease, disorder, and/or condition; amelioration or palliation of the state of a disease, disorder, and/or condition; and remission (whether partial or total), whether detectable or undetectable; and/or overall survival.

As used herein, “prevention” of or “preventing” a disorder or condition refers to reduction of or reducing the occurrence of the disorder or condition in a treated sample relative to an untreated control sample, and includes delaying onset, progression, or reduction of severity of one or more symptoms of the disorder or condition relative to the untreated control sample.

The terms “patient,” “subject,” “individual,” and the like, as used herein, are interchangeable and refer to any animal, which may be a human or a non-human animal.

As will be understood by one of ordinary skill in the art, when disclosed herein, each range includes all possible subranges as well as individual numerical values within that range. For example, a range of “1.0 to 5.0” includes and would be understood to specifically disclose subranges such as “1.0 to 3.0,” “1.5 to 3.7,” “2.1 to 4.3,” etc., as well as all individual numbers within the disclosed range, for example, 1.0, 1.1, 1.2, 1.3, etc.

As used herein, a formulation is “stable” if, after being autoclaved for 30 minutes at 124° C. and thereafter cooled to room temperature, there is (a) no visible darkening of the color of the formulation or any ingredient therein, (b) no drop in pH of the formulation of more than 0.25 pH units, and (c) no increase in the osmolality of the formulation of more than 15 mOsm. As used herein, visible darkening is determined according to USP <631>, if the color of the pre-autoclaved and post-autoclaved samples are indiscernable in color (by method I) or exhibiting a delta E* of <3 (by method II), then no visible change in color has occurred. As used herein, pH is measured according to USP <791> and osmolality is measured according to USP <785>.

As used herein, a formulation is “shelf stable” if, after being stored at 20-25° C. for 24 months, there is (a) no drop in pH of the formulation of more than 1.0 pH units and (b) no increase in the osmolality of the formulation of more than 50 mOsm. As used herein, pH is measured according to USP <791> and osmolality is measured according to USP <785>. As used herein, a formulation is “free of” a compound when said compound is not added during manufacture of the formulation.

As noted above, the present disclosure is related to pre-moistened wipes and cleansing formulations, the formulations comprising at least one surfactant chosen from decyl glucoside and sorbitan oleate decylglucoside crosspolymer (sugamulse D9). In some embodiments, the formulation further comprises at least one natural humectant. In some embodiments, the formulation further comprises at least one plant extract. In some embodiments, the formulation further comprises at least one buffer.

In some embodiments, the formulation comprises decyl glucoside and sorbitan oleate decylglucoside crosspolymer (sugamulse D9) and further comprises sodium hyaluronate and at least one natural humectant having antioxidant activity. In some embodiments, the at least one natural humectant having antioxidant activity is chosen from erythritol and ectoine. In some embodiments, the formulation comprises decyl glucoside, sorbitan oleate decylglucoside crosspolymer (sugamulse D9), sodium hyaluronate, at least one natural humectant having antioxidant activity, and propanediol.

In some embodiments, the formulation further comprises at least one buffer. In some embodiments, the at least one buffer is chosen from citrate-phosphate buffers. In some embodiments, at least one salt is used to form the citrate-phosphate buffers. In some embodiments, at least one salt is chosen from sodium chloride, potassium chloride, sodium phosphate dibasic, sodium citrate dihydrate, and citric acid.

In some embodiments, the at least one buffer provides the formulation with a pH of 6-8. In some embodiments, the at least one buffer provides the formulation with a pH of 7-8. In some embodiments, the at least one buffer provides the formulation with a pH of 6. In some embodiments, the at least one buffer provides the formulation with a pH of 7. In some embodiments, the at least one buffer provides the formulation with a pH of 8.In some embodiments, the at least one buffer provides the formulation with a pH of 6.0, 6.1, 6.2, 6.3, 6.4, 6.5, 6.6, 6.7, 6.8, or 6.9. In some embodiments, the at least one buffer provides the formulation with a pH of 7.0, 7.1, 7.2, 7.3, 7.4, 7.5, 7.6, 7.7, 7.8, 7.9, or 8.0. In some embodiments, the pH of the formulation may be measured before being placed into the packaging. In some embodiments, the pH of the formulation may be measured after expressing it from a package containing a pre-moistened wipe.

In some embodiments, the formulation has an osmolality of 230-450 mOsm/kg. In some embodiments, the formulation has an osmolality of 250-450 mOsm/kg. In some embodiments, the formulation has an osmolality of 250 mOsm/kg. In some embodiments, the formulation has an osmolality of 270 mOsm/kg. In some embodiments, the formulation has an osmolality of 290 mOsm/kg. In some embodiments, the formulation has an osmolality of 310 mOsm/kg. In some embodiments, the formulation has an osmolality of 330 mOsm/kg. In some embodiments, the formulation has an osmolality of 350 mOsm/kg. In some embodiments, the formulation has an osmolality of 370 mOsm/kg. In some embodiments, the formulation has an osmolality of 390 mOsm/kg. In some embodiments, the formulation has an osmolality of 410 mOsm/kg. In some embodiments, the formulation has an osmolality of 430 mOsm/kg. In some embodiments, the formulation has an osmolality of 450 mOsm/kg. In some embodiments, the osmolality of the formulation is measured before being placed into packaging. In some embodiments, the osmolality of the formulation is measured after expressing it from a package containing a pre-moistened wipe.

In some embodiments, the formulations disclosed herein are stable. In some embodiments, the formulations are shelf-stable.

In some embodiments, after being stored at 20-25° C. for 24 months, the pH of the formulation does not drop more than 0.75 pH units. In some embodiments, after being stored at 20-25° C. for 24 months, the pH of the formulation does not drop more than 0.6 pH units. In some embodiments, after being stored at 20-25° C. for 24 months, the pH of the formulation does not drop more than 0.5 pH units. In some embodiments, after being stored at 20-25° C. for 24 months, the pH of the formulation does not drop more than 0.4 pH units. In some embodiments, after being stored at 20-25° C. for 24 months, the pH of the formulation does not drop more than 0.3 pH units. In some embodiments, after being stored at 20-25° C. for 24 months, the pH of the formulation does not drop more than 0.25 pH units. In some embodiments, after being stored at 20-25° C. for 24 months, the pH of the formulation does not drop more than 0.2 pH units. In some embodiments, after being stored at 20-25° C. for 24 months, the pH of the formulation does not drop more than 0.1 pH units.

In some embodiments, after being stored at 20-25° C. for 24 months, the osmolality of the formulation does not increase more than 50 mOsm. In some embodiments, after being stored at 20-25° C. for 24 months, the osmolality of the formulation does not increase more than 40 mOsm. In some embodiments, after being stored at 20-25° C. for 24 months, the osmolality of the formulation does not increase more than 30 mOsm. In some embodiments, after being stored at 20-25° C. for 24 months, the osmolality of the formulation does not increase more than 25 mOsm. In some embodiments, after being stored at 20-25° C. for 24 months, the osmolality of the formulation does not increase more than 20 mOsm. In some embodiments, after being stored at 20-25° C. for 24 months, the osmolality of the formulation does not increase more than 15 mOsm.

In some embodiments, there is provided a wipe for the eye area, comprising a pad which is pre-moistened with a cleansing formulation. In some embodiments, the pre-moistened pad is sealed in a package. In some embodiments, the package is a foil-foil package. In some embodiments, the package is a laminate foil pouch. In some embodiments, the package is a laminate foil pouch sealed by at least one adhesive. In some embodiments, the package is a laminate foil pouch sealed by at least one hot melt adhesive. In some embodiments, the wipe is individually foil wrapped.

In some embodiments, a cleansing formulation in a sealed package. In some embodiments, the formulation is sealed in a plastic bottle. In some embodiments, the plastic bottle is made of polyethylene. In some embodiments, the plastic bottle is made of polypropylene. In some embodiments, the plastic bottle is made of polyethylene terephthalate (PET).

In some embodiments, the wipe is pre-moistened with 1-2 g of formulation. In some embodiments, the wipe is pre-moistened with 1.5 g of formulation. In some embodiments, the formulation comprises decyl glucoside and/or sorbitan oleate decylglucoside crosspolymer (sugamulse D9). In some embodiments, decyl glucoside and sorbitan oleate decylglucoside crosspolymer are naturally derived.

In some embodiments, the formulation is sterilized by filtration, autoclaving, gamma irradiation and/or electron-beam. In some embodiments, the formulation is sterilized by filtration. In some embodiments, the formulation is sterilized by autoclaving. In some embodiments, the formulation is sterilized by filtration and autoclaving. In some embodiments, the formulation is sterilized by gamma irradiation. In some embodiments, the formulation is sterilized by electron-beam.

In some embodiments, the formulation is sterilized by irradiation with low dose gamma radiation. In some embodiments, the formulation is sterilized at gamma irradiation at 15-55 kGy. In some embodiments, the formulation is sterilized at gamma irradiation at 20-50 kGy. In some embodiments, the formulation is sterilized at gamma irradiation at 25-50 kGy. In some embodiments, the formulation is sterilized at gamma irradiation at 25-40 kGy. In some embodiments, the formulation is sterilized at gamma irradiation at 40-50 kGy.

In some embodiments, the formulation comprises decyl glucoside and/or sorbitan oleate decylglucoside crosspolymer (sugamulse D9). In some embodiments, the formulation further comprises at least one plant extract. In some embodiments, the at least one plant extract is aloe barbadensis leaf juice. In some embodiments, the at least one plant extract is dandelion root (taraxacum officinale) extract. In some embodiments, the at least one plant extract is kelp (ascophyllum nodosum) extract. In some embodiments, the at least one plant extract is eyebright (euphrasia officinalis) extract. In some embodiments, the at least one plant extract is caffeine. In some embodiments, the at least one plant extract is white mulberry (morus alba) bark extract. In some embodiments, the at least one plant extract is licorice root (glycyrrhiza glabra) extract. In some embodiments, the at least one plant extract is cucumber fruit (cucumis sativus). In some embodiments, the at least one plant extract is menthol. In some embodiments, the at least one plant extract is comprised in glycerine. In some embodiments, the at least one plant extract is comprised in butylene glycol. In some embodiments, the butylene glycol is naturally derived. In some embodiments, the butylene glycol is made from fermentation of natural, non-GMO sugar.

In some embodiments, the formulation further comprises at least one vitamin. In some embodiments, the at least one vitamin is d-panthenol (pro-vitamin B). In some embodiments, the at least one vitamin is tocopherol (vitamin E). In some embodiments, the at least one vitamin is sodium ascorbyl phosphate (vitamin C). In some embodiments, the at least one vitamin is niacinamide (vitamin B3). In some embodiments, the at least one vitamin is d-panthenol (pro-vitamin B5). In some embodiments, the at least one vitamin is d-biotin (vitamin B7).

In some embodiments, the formulation further comprises at least one antimicrobial agent. In some embodiments, the at least one antimicrobial agent is caprylyl glycol. In some embodiments, the at least one antimicrobial agent is phenylpropanol. In some embodiments, the at least one antimicrobial agent is 1,2-hexanediol. In some embodiments, the at least one antimicrobial agent is caprylyl glycol and phenylpropanol. In some embodiments, the at least one antimicrobial agent is 1,2-hexanediol and caprylyl glycol. In some embodiments, the at least one antimicrobial agent is alexidine chloride. In some embodiments, the formulation is free from antimicrobial agents such as hypochlorous acid or tea tree oil.

In some embodiments, disclosed herein are formulations comprising decyl glucoside; sugamulse D9 (sorbitan oleate decylglucoside crosspolymer); propanediol; licorice root (glycyrrhiza glabra) extract; and ectoine, erithrytol, or a mixture of the two; and having a pH of 6-8. In some embodiments, the formulations have a pH of 7-8. In some embodiments, the formulations have a pH of 7.4-7.6. In some embodiments, the formulations further comprise sodium hyaluronate.

In some embodiments, the formulations disclosed herein are free of parabens, thiazolinones, and formaldehyde-donor preservatives. In some embodiments, formulations disclosed herein are free of polyethylene glycol (PEG) and PEG-containing ingredients. In some embodiments, formulations disclosed herein are preservative free. In some embodiments, formulations disclosed herein are sterile and preservative free. In some embodiments, formulations disclosed herein are micellar cleansing formulas. In some embodiments, formulations disclosed herein are oil-free formulas. In some embodiments, formulations disclosed herein are leave-on formulas that do not require rinsing. In some embodiments, formulations disclosed herein are pH-balanced and non-irritating.

In some embodiments, the formulations disclosed herein comprise water, aloe barbadensis leaf juice, decyl glucoside, sorbitan oleate decylglucoside crosspolymer, propanediol, glycerin, erythritol, sodium chloride, sodium hyaluronate, ectoine, d-panthenol (pro-vitamin B5), potassium chloride, sodium phosphate dibasic, sodium citrate, licorice root (glycyrrhiza glabra) extract, cucumber fruit (cucumber sative) extract, dandelion root (taraxacum officinale) extract, citric acid, and may optionally further comprise 1,2-hexanediol and/or caprylyl glycol. In some embodiments, the formulations disclosed herein comprise water, propanediol, decyl glucoside, sorbitan oleate decylglucoside crosspolymer, butylene glycol, ectoine, sodium hyaluronate, d-panthenol (pro-vitamin B5), sodium ascorbyl phosphate (vitamin C), d-biotin (vitamin B7), niacinamide (vitamin B3), potassium chloride, sodium phosphate dibasic, sodium citrate, licorice root (glycyrrhiza glabra) extract, cucumber fruit (cucumis sative) extract, white mulberry (morus alba) bark extract, eyebright (euphrasia officinalis) extract, kelp (ascophyllum nodosum) extract, menthol, caffeine, and citric acid, and may optionally further comprise caprylyl glycol, 1,2-hexanediol, and/or alexidine chloride.

Also disclosed herein are formulations comprising 0.01 wt% to 0.25 wt% sodium hyaluronate; 0.50 wt% to 1.10 wt% propanediol; 0.01 wt% to 0.25 wt% ectoine; 0 wt% to 0.50 wt% erythritol; 0 wt% to 0.25 wt% licorice root (glycyrrhiza glabra) extract; 0.1 wt% to 1 wt% sorbitan oleate decylglucoside crosspolymer; and 0.06 wt% to 1 wt% decyl glucoside; and having a pH of 6-8. In some embodiments, the formulations have a pH of 7-8. In some embodiments, the formulations have a pH of 7.4-7.6.

Also disclosed herein are formulations comprising 0.01 wt% to 0.24 wt% sodium hyaluronate; 0.55 wt% to 1.05 wt% propanediol; 0.01 wt% to 0.20 wt% ectoine; 0.01 wt% to 0.30 wt% erythritol; 0.5 to 1 wt% sorbitan oleate decylglucoside crosspolymer; 0.5 wt% to 1 wt% decyl glucoside; and at least one plant extract chosen from 0.01 wt% to 0.20 wt% licorice root (glycyrrhiza glabra) extract, 0.01 wt% to 0.20% wt% aloe barbadensis leaf juice (concentrate 200:1), 0.01 wt% to 0.20 wt% dandelion root (taraxacum officinale) extract, 0.01 wt% to 0.20 wt% white mulberry (morus alba) bark extract, 0.01 wt% to 0.20 wt% cucumber fruit (cucumis sative) extract, 0.01 wt% to 0.20 wt% kelp (ascophyllum nodosum) extract, 0.01 wt% to 0.20 wt% eyebright (euphrasia officinalis) extract, 0.0010 wt% to 0.0030 wt% L-menthol, and 0.005 wt% to 0.20 wt% caffeine; and having a pH of 6-8. In some embodiments, the formulations have a pH of 7-8. In some embodiments, the formulations have a pH of 7.4-7.6.

Also disclosed herein are formulations comprising 0.01 wt% to 0.24 wt% sodium hyaluronate; 0.55 wt% to 1.05 wt% propanediol; 0.01 wt% to 0.20 wt% ectoine; 0.01 wt% to 0.20 wt% d-panthenol; 0.01 wt% to 0.20 wt% sodium ascorbyl phosphate; 0.01 wt% to 0.20 wt% licorice root (glycyrrhiza glabra) extract; 0.01 wt% to 0.20 wt% cucumber fruit (cucumis sative) extract, 0.01 wt% to 0.20 wt% dandelion root (taraxacum officinale) extract; 0.01 wt% to 0.20 wt% white mulberry (morus alba) bark extract; 0.01 wt% to 0.20 wt% kelp (ascophyllum nodosum) extract; 0.01 wt% to 0.20 wt% eyebright (euphrasia officinalis) extract; 0.0010 wt% to 0.0030 wt% L-menthol; 0.005 wt% to 0.20 wt% caffeine; 0.5 wt% to 1 wt% sorbitan oleate decylglucoside crosspolymer; and 0.5 wt% to 1 wt% decyl glucoside; and having a pH of 6-8. In some embodiments, the formulations have a pH of 7-8. In some embodiments, the formulations have a pH of 7.4-7.6. In some embodiments, said formulations further comprise at least one antimicrobial agent, for example at least one antimicrobial agent chosen from 0.01 wt% to 0.5 wt% caprylyl glycol and phenylpropanol, 0.01 wt% to 0.5 wt% 1,2-hexanediol and caprylyl glycol, and 0.01 to 2.0 wt% alexidine chloride (0.5% solution).

Also disclosed herein are formulations comprising 0.01 wt% to 0.20 wt% sodium hyaluronate; 0.55 wt% to 1.05 wt% propanediol; 0.01 wt% to 0.20 wt% ectoine; 0.01 wt% to 0.50 wt% erythritol; 0.01 wt% to 0.20 wt% d-panthenol; 0.01 wt% to 0.20% wt% aloe barbadensis leaf juice (concentrate 200:1); 0.01 wt% to 0.20 wt% licorice root (glycyrrhiza glabra) extract; 0.01 wt% to 0.20 wt% cucumber fruit (cucumis sative) extract; 0.01 wt% to 0.20 wt% dandelion root (taraxacum officinale) extract; 0.5 wt% to 1 wt% sorbitan oleate decylglucoside crosspolymer; and 0.5 wt% to 1 wt% decyl glucoside; and having a pH of 6-8. In some embodiments, the formulations have a pH of 7-8. In some embodiments, the formulations have a pH of 7.4-7.6.

Also disclosed herein are formulations comprising 0.01 wt% to 0.20 wt% sodium hyaluronate; 0.55 wt% to 1.05 wt% propanediol; 0.01 wt% to 0.50 wt% ectoine; 0.01 wt% to 1.05 wt% niacinamide; 0.01 wt% to 1.05% sodium ascorbyl phosphate; 0.01 wt% to 0.20 wt% d-panthenol; 0.01 wt% to 0.20 wt% licorice root (glycyrrhiza glabra) extract; 0.01 wt% to 0.20 wt% cucumber fruit (cucumis sative) extract; 0.01 wt% to 0.20 wt% eyebright (euphrasia Officinalis) extract; 0.5 wt% to 1 wt% sorbitan oleate decylglucoside crosspolymer; and 0.5 wt% to 1 wt% decyl glucoside; has and having a pH of 6-8. In some embodiments, the formulations have a pH of 7-8. In some embodiments, the formulations have a pH of 7.4-7.6.

In some embodiments, the formulation comprises 0.10 wt% to 0.24 wt% sodium hyaluronate; 0.55 wt% to 1.05 wt% propanediol; 0.08 wt% to 0.20 wt% ectoine; 0.01 wt% to 0.20 wt% d-panthenol (pro-vitamin B5); 0.015 wt% to 0.025 wt% citric acid monohydrate; 0.10 wt% to 1.0 wt% sorbitan oleate decylglucoside crosspolymer; and 0.06 wt% to 1.0 wt% decyl glucoside. In some embodiments, the formulation further comprises 0.01 wt% to 0.30 wt% erythritol; 0.01 wt% to 0.05 wt% d-biotin (vitamin B7); 0.01 wt% 0.95 wt% niacinamide (vitamin B3); 0.01 wt% to 0.20 wt% sodium ascorbyl phosphate (vitamin C); 0.01 wt% to 0.20 wt% sodium chloride; 0.01 wt% to 0.10 wt% potassium chloride; 0.01 wt% to 0.10 wt% sodium phosphate dibasic; 0.01 wt% to 0.10 wt% sodium citrate dihydrate; 0.01 wt% to 0.20 wt% 1,2-hexanediol and caprylyl glycol; 0.01 wt% to 2.0 wt% alexidine chloride, 0.5% solution; 0.01 wt% to 0.20 wt% aloe barbadensis leaf juice (concentrate 200:1); 0.01 wt% to 0.20 wt% licorice root (glycyrrhiza glabra) extract in glycerin or butylene glycol; 0.01 wt% to 0.20 wt% dandelion root (taraxacum officinale) extract in glycerin; 0.01 wt% to 0.10 wt% white mulberry (morus alba) bark extract in butylene glycol; 0.01 wt% to 0.20 wt% cucumber fruit (cucumis sative) extract in glycerin; 0.01 wt% to 0.20 wt% kelp (ascophyllum nodosum) extract in butylene glycol; 0.01 wt% to 0.20 wt% eyebright (euphrasia officinalis) extract in glycerin; 0.01 wt% to 0.02 wt% caffeine; and 0.01 wt% to 0.02 wt% L-menthol.

In some embodiments, the formulation comprises 0.12 wt% sodium hyaluronate; 0.80 wt% propanediol; 0.15 wt% ectoine; 0.15 wt% erythritol; 0.10 wt% d-panthenol (pro-vitamin B5); 0.15 wt% sodium chloride; 0.10 wt% potassium chloride; 0.10 wt% sodium phosphate dibasic; 0.10 wt% sodium citrate dihydrate; 0.015 wt% citric acid monohydrate; 0.01 wt% aloe barbadensis leaf juice (concentrate 200:1); 0.10 wt% licorice root (glycyrrhiza glabra) extract in glycerin; 0.05 wt% dandelion root (taraxacum officinale) extract in glycerin; 0.75 wt% sorbitan oleate decylglucoside crosspolymer; and 0.75 wt% decyl glucoside.

In some embodiments, the formulation comprises 0.12 wt% sodium hyaluronate; 0.80 wt% propanediol; 0.20 wt% ectoine; 0.05 wt% d-panthenol (pro-vitamin B5); 0.05 wt% d-biotin (vitamin B7); 0.10 wt% niacinamide (vitamin B3); 0.05 wt% sodium ascorbyl phosphate (vitamin C); 0.10 wt% potassium chloride; 0.10 wt% sodium phosphate dibasic; 0.10 wt% sodium citrate dihydrate; 0.015 wt% citric acid monohydrate; 0.20 wt% 1,2-hexanediol and caprylyl glycol; 2.0 wt% alexidine chloride, 0.5% solution; 0.10 wt% licorice root (glycyrrhiza glabra) extract in butylene glycol; 0.10 wt% white mulberry (morus alba) bark extract in butylene glycol; 0.05 wt% kelp (ascophyllum nodosum) extract in butylene glycol; 0.05 wt% eyebright (euphrasia officinalis) extract in glycerin; 0.02 wt% caffeine; 0.75 wt% sorbitan oleate decylglucoside crosspolymer; and 0.75 wt% decyl glucoside.

In some embodiments, the formulation comprises 0.12 wt% sodium hyaluronate; 0.80 wt% propanediol; 0.20 wt% ectoine; 0.10 wt% d-panthenol (pro-vitamin B5); 0.05 wt% niacinamide (vitamin B3); 0.10 wt% sodium ascorbyl phosphate (vitamin C); 0.05 wt% potassium chloride; 0.10 wt% sodium phosphate dibasic; 0.10 wt% sodium citrate dihydrate; 0.025 wt% citric acid monohydrate; 2.0 wt% alexidine chloride, 0.5% solution; 0.10 wt% licorice root (glycyrrhiza glabra) extract in glycerin; 0.10 wt% cucumber fruit (cucumis sative) extract in glycerin; 0.05 wt% kelp (ascophyllum nodosum) extract in butylene glycol; 0.05 wt% eyebright (euphrasia officinalis) extract in glycerin; 0.0025 wt% L-menthol; 0.02 wt% caffeine; 0.75 wt% sorbitan oleate decylglucoside crosspolymer; and 0.75 wt% decyl glucoside.

In some embodiments, the formulation comprises 0.12 wt% sodium hyaluronate; 0.80 wt% propanediol; 0.20 wt% ectoine; 0.20 wt% erythritol; 0.20 wt% d-panthenol (pro-vitamin B5); 0.15 wt% sodium chloride; 0.10 wt% potassium chloride; 0.10 wt% sodium phosphate dibasic; 0.10 wt% sodium citrate dihydrate; 0.015 wt% citric acid monohydrate; 0.06 wt% aloe barcadensis leaf juice (concentrate 200:1); 0.01 wt% licorice root (glycyrrhiza glabra) extract in glycerin; 0.18 wt% dandelion root (taraxacum officinale) extract in glycerin; 0.90 wt% sorbitan oleate decylglucoside crosspolymer; and 0.90 wt% decyl glucoside.

In some embodiments, the formulation comprises 0.18 wt% sodium hyaluronate; 0.60 wt% propanediol; 0.10 wt% ectoine; 0.10 wt% erythritol; 0.15 wt% d-panthenol (pro-vitamin B5); 0.20 wt% sodium chloride; 0.05 wt% potassium chloride; 0.10 wt% sodium phosphate dibasic; 0.10 wt% sodium citrate dihydrate; 0.015 wt% citric acid monohydrate; 0.10 wt% aloe barcadensis leaf juice (concentrate 200:1); 0.15 wt% licorice root (glycyrrhiza glabra) extract in glycerin; 0.02 wt% dandelion root (taraxacum officinale) extract in glycerin; 0.5 wt% sorbitan oleate decylglucoside crosspolymer; and 0.5 wt% decyl glucoside.

In some embodiments, the formulation comprises 0.24 wt% sodium hyaluronate; 0.80 wt% propanediol; 0.10 wt% ectoine; 0.15 wt% erythritol; 0.15 wt% d-panthenol (pro-vitamin B5); 0.20 wt% sodium chloride; 0.05 wt% potassium chloride; 0.10 wt% sodium phosphate dibasic; 0.10 wt% sodium citrate dihydrate; 0.015 wt% citric acid monohydrate; 0.01 wt% aloe barcadensis leaf juice (concentrate 200:1); 0.10 wt% licorice root (glycyrrhiza glabra) extract in glycerin; 0.20 wt% dandelion root (taraxacum officinale) extract in glycerin; 1.0 wt% sorbitan oleate decylglucoside crosspolymer; and 1.0 wt% decyl glucoside.

In some embodiments, the formulation comprises 0.24 wt% sodium hyaluronate; 1.05 wt% propanediol; 0.15 wt% ectoine; 0.15 wt% erythritol; 0.05 wt% d-panthenol (pro-vitamin B5); 0.20 wt% sodium chloride; 0.05 wt% potassium chloride; 0.10 wt% sodium phosphate dibasic; 0.10 wt% sodium citrate dihydrate; 0.015 wt% citric acid monohydrate; 0.20 wt% aloe barcadensis leaf juice (concentrate 200:1); 0.15 wt% licorice root (glycyrrhiza glabra) extract in glycerin; 0.05 wt% dandelion root (taraxacum officinale) extract in glycerin; 0.5 wt% sorbitan oleate decylglucoside crosspolymer; and 1.0 wt% decyl glucoside.

In some embodiments, the formulation comprises 0.15 wt% sodium hyaluronate; 0.75 wt% propanediol; 0.20 wt% ectoine; 0.30 wt% erythritol; 0.05 wt% d-panthenol (pro-vitamin B5); 0.20 wt% sodium chloride; 0.05 wt% potassium chloride; 0.10 wt% sodium phosphate dibasic; 0.10 wt% sodium citrate dihydrate; 0.015 wt% citric acid monohydrate; 0.10 wt% aloe barcadensis leaf juice (concentrate 200:1); 0.20 wt% licorice root (glycyrrhiza glabra) extract in glycerin; 0.15 wt% dandelion root (taraxacum officinale) extract in glycerin; 1.0 wt% sorbitan oleate decylglucoside crosspolymer; and 0.5 wt% decyl glucoside.

In some embodiments, the formulation comprises 0.15 wt% sodium hyaluronate; 0.75 wt% propanediol; 0.10 wt% ectoine; 0.20 wt% erythritol; 0.20 wt% d-panthenol (pro-vitamin B5); 0.15 wt% sodium chloride; 0.10 wt% potassium chloride; 0.10 wt% sodium phosphate dibasic; 0.10 wt% sodium citrate dihydrate; 0.015 wt% citric acid monohydrate; 0.05 wt% aloe barcadensis leaf juice (concentrate 200:1); 0.15 wt% licorice root (glycyrrhiza glabra) extract in glycerin; 0.10 wt% dandelion root (taraxacum officinale) extract in glycerin; 0.65 wt% sorbitan oleate decylglucoside crosspolymer; and 0.85 wt% decyl glucoside.

In some embodiments, the formulation comprises 0.15 wt% sodium hyaluronate; 0.80 wt% propanediol; 0.10 wt% ectoine; 0.15 wt% d-panthenol (pro-vitamin B5); 0.80 wt% niacinamide (vitamin B3); 0.20 wt% sodium ascorbyl phosphate (vitamin C); 0.025 wt% citric acid monohydrate; 2.0 wt% alexidine chloride, 0.5% solution; 0.15 wt% licorice root (glycyrrhiza glabra) extract in glycerin; 0.20 wt% cucumber fruit (cucumis sative) extract in glycerin; 0.05 wt% eyebright (euphrasia officinalis) extract in glycerin; 0.80 wt% sorbitan oleate decylglucoside crosspolymer; and 0.70 wt% decyl glucoside.

In some embodiments, the formulation comprises 0.15 wt% sodium hyaluronate; 0.60 wt% propanediol; 0.20 wt% ectoine; 0.15 wt% d-panthenol (pro-vitamin B5); 0.05 wt% d-biotin (vitamin B7); 0.10 wt% niacinamide (vitamin B3); 0.05 wt% sodium ascorbyl phosphate (vitamin C); 0.10 wt% potassium chloride; 0.10 wt% sodium phosphate dibasic; 0.10 wt% sodium citrate dihydrate; 0.015 wt% citric acid monohydrate; 0.20 wt% 1,2-hexanediol and caprylyl glycol; 2.0 wt% alexidine chloride, 0.5% solution; 0.10 wt% licorice root (glycyrrhiza glabra) extract in butylene glycol; 0.10 wt% white mulberry (morus alba) bark extract in butylene glycol; 0.15 wt% cucumber fruit (cucumis sative) extract in glycerin; 0.01 wt% eyebright (euphrasia officinalis) extract in glycerin; 0.02 wt% L-menthol; 1.0 wt% sorbitan oleate decylglucoside crosspolymer; and 1.0 wt% decyl glucoside.

In some embodiments, the formulation comprises 0.15 wt% sodium hyaluronate; 1.05 wt% propanediol; 0.20 wt% ectoine; 0.01 wt% d-panthenol (pro-vitamin B5); 0.05 wt% niacinamide (vitamin B3); 0.10 wt% sodium ascorbyl phosphate (vitamin C); 0.05 wt% potassium chloride; 0.10 wt% sodium phosphate dibasic; 0.10 wt% sodium citrate dihydrate; 0.025 wt% citric acid monohydrate; 2.0 wt% alexidine chloride, 0.5% solution; 0.10 wt% licorice root (glycyrrhiza glabra) extract in glycerin; 0.10 wt% cucumber fruit (cucumis sative) extract in glycerin; 0.20 wt% kelp (ascophyllum nodosum) extract in butylene glycol; 0.10 wt% eyebright (euphrasia officinalis) extract in glycerin; 0.02 wt% caffeine; 0.75 wt% sorbitan oleate decylglucoside crosspolymer; and 1.0 wt% decyl glucoside.

In some embodiments, the formulation comprises 0.12 wt% sodium hyaluronate; 0.80 wt% propanediol; 0.20 wt% ectoine; 0.10 wt% d-panthenol (pro-vitamin B5); 0.95 wt% niacinamide (vitamin B3); 0.20 wt% sodium ascorbyl phosphate (vitamin C); 0.025 wt% citric acid monohydrate; 2.0 wt% alexidine chloride, 0.5% solution; 0.10 wt% licorice root (glycyrrhiza glabra) extract in glycerin; 0.10 wt% cucumber fruit (cucumis sative) extract in glycerin; 0.05 wt% eyebright (euphrasia officinalis) extract in glycerin; 0.75 wt% sorbitan oleate decylglucoside crosspolymer; and 0.75 wt% decyl glucoside.

In some embodiments, the formulation comprises 0.18 wt% sodium hyaluronate; 0.60 wt% propanediol; 0.08 wt% ectoine; 0.10 wt% d-panthenol (pro-vitamin B5); 0.70 wt% niacinamide (vitamin B3); 0.20 wt% sodium ascorbyl phosphate (vitamin C); 0.025 wt% citric acid monohydrate; 2.0 wt% alexidine chloride, 0.5% solution; 0.15 wt% licorice root (glycyrrhiza glabra) extract in glycerin; 0.05 wt% cucumber fruit (cucumis sative) extract in glycerin; 0.20 wt% eyebright (euphrasia officinalis) extract in glycerin; 0.5 wt% sorbitan oleate decylglucoside crosspolymer; and 0.5 wt% decyl glucoside.

In some embodiments, the formulation comprises 0.18 wt% sodium hyaluronate; 0.80 wt% propanediol; 0.10 wt% ectoine; 0.15 wt% d-panthenol (pro-vitamin B5); 0.90 wt% niacinamide (vitamin B3); 0.20 wt% sodium ascorbyl phosphate (vitamin C); 0.025 wt% citric acid monohydrate; 1.0 wt% alexidine chloride, 0.5% solution; 0.10 wt% licorice root (glycyrrhiza glabra) extract in glycerin; 0.15 wt% cucumber fruit (cucumis sative) extract in glycerin; 0.10 wt% eyebright (euphrasia officinalis) extract in glycerin; 0.2 wt% sorbitan oleate decylglucoside crosspolymer; and 0.6 wt% decyl glucoside.

In some embodiments, the formulation comprises 0.18 wt% sodium hyaluronate; 1.05 wt% propanediol; 0.15 wt% ectoine; 0.08 wt% d-panthenol (pro-vitamin B5); 0.10 wt% niacinamide (vitamin B3); 0.20 wt% sodium ascorbyl phosphate (vitamin C); 0.025 wt% citric acid monohydrate; 1.5 wt% alexidine chloride, 0.5% solution; 0.15 wt% licorice root (glycyrrhiza glabra) extract in glycerin; 0.02 wt% cucumber fruit (cucumis sative) extract in glycerin; 0.18 wt% eyebright (euphrasia officinalis) extract in glycerin; 0.1 wt% sorbitan oleate decylglucoside crosspolymer; and 1.0 wt% decyl glucoside.

In some embodiments, the formulation comprises 0.1 wt% sodium hyaluronate; 0.75 wt% propanediol; 0.20 wt% ectoine; 0.05 wt% d-panthenol (pro-vitamin B5); 0.20 wt% niacinamide (vitamin B3); 0.20 wt% sodium ascorbyl phosphate (vitamin C); 0.025 wt% citric acid monohydrate; 1.0 wt% alexidine chloride, 0.5% solution; 0.20 wt% licorice root (glycyrrhiza glabra) extract in glycerin; 0.18 wt% cucumber fruit (cucumis sative) extract in glycerin; 0.18 wt% eyebright (euphrasia officinalis) extract in glycerin; 0.2 wt% sorbitan oleate decylglucoside crosspolymer; and 0.6 wt% decyl glucoside.

In some embodiments, the formulation comprises 0.10 wt% sodium hyaluronate; 0.55 wt% propanediol; 0.12 wt% ectoine; 0.10 wt% d-panthenol (pro-vitamin B5); 0.45 wt% niacinamide (vitamin B3); 0.20 wt% sodium ascorbyl phosphate (vitamin C); 0.025 wt% citric acid monohydrate; 1.5 wt% alexidine chloride, 0.5% solution; 0.18 wt% licorice root (glycyrrhiza glabra) extract in glycerin; 0.10 wt% cucumber fruit (cucumis sative) extract in glycerin; 0.01 wt% eyebright (euphrasia officinalis) extract in glycerin; 0.3 wt% sorbitan oleate decylglucoside crosspolymer; and 0.06 wt% decyl glucoside.

Combination of the disclosed formulations may advantageously produce one or more of the following effects: (1) additive and/or synergistic benefits; (2) reduction of the side effects and/or adverse effects associated with use of a prescription medicine in the absence of the formulations disclosed herein; and/or (3) the ability to lower the dosage of the prescription medicine in comparison to the amount of prescription medicine needed in the absence of the formulation disclosed herein.

The formulation disclosed herein may be prepared according to any known method for the manufacture of cosmetic formulations or preparations. As will be appreciated by those of ordinary skill in the art, a number of methods are known.

In some embodiments, the formulations disclosed herein are in the form of pre-moistened sterile wipes or cleansing pads, optionally comprising at least one antimicrobial agent.

In some embodiments, the formulations disclosed herein are non-sterile liquid multi-use formulations, comprising at least one antimicrobial agent, and are suitable for use with wipes, cloths, tissues, pads, or otherwise suitable material. In some embodiments, the wipes are formed from an absorbent material, and include pads, swabs, tissues, cottonwool, washcloths, and fiber applicators of any kind that are able to absorb the formulation and then be applied to the skin surface to clean, condition, moisturize, lubricate, soothe, calm, refresh, whiten or brighten, and/or remove material on the skin.

In some embodiments, the formulations disclosed herein are useful for cleansing, cleaning, conditioning, moisturizing, lubricating, soothing, calming, refreshing, whitening, lightening, and/or brightening the eyelid, periocular, and ocular surface tissues. In some embodiments, formulations disclosed herein are useful for treating, improving, and/or ameliorating inflammation; removing irritants, makeup, debris, crusting, excess oil and/or secretions; maintaining eyelid hygiene; and/or treating, improving, and/or ameliorating blepharitis and symptoms thereof. In some embodiments, formulations disclosed herein are suitable for adults, children, and contact-lens wearers.

Accordingly, provided herein are methods of cleansing, cleaning, conditioning, moisturizing, lubricating, soothing, calming, refreshing, whitening, lightening, and/or brightening the eyelid, periocular, and/or ocular surface tissues, comprising applying to said eyelid, periocular, and/or ocular surface tissues a formulation or pre-moistened wipe as disclosed herein. In some embodiments, provided herein are methods of treating, improving, and/or ameliorating inflammation; removing irritants, makeup, debris, crusting, and/or excess oil and/or secretions; maintaining eyelid hygiene; and/or treating, improving, or ameliorating blepharitis and symptoms thereof. The methods comprise applying to eyelid, periocular, and/or ocular surface tissues a formulation or pre-moistened wipe as disclosed herein.

### PROPHETIC EXAMPLES

Formulations of the present disclosure can be prepared with ingredients as listed in the following tables.

TABLE 1A

| Formulation component | Ingredient | Formulation (Wt%) | | | | | | | | |
|---|---|---|---|---|---|---|---|---|---|---|
| | | A | B | C | D | E | F | G | H | I |
| Polymers /Viscosity-increasing Agent | Sodium Hyaluronate | 0.12 | 0.12 | 0.12 | 0.12 | 0.18 | 0.24 | 0.24 | 0.15 | 0.15 |
| Humectants | Propanediol | 0.80 | 0.80 | 0.80 | 0.80 | 0.60 | 0.80 | 1.05 | 0.75 | 0.75 |
| | Glycerin | (in extracts) | -- | (in extracts) | (in extracts) | (in extracts) | (in extracts) | (in extracts) | (in extracts) | (in extracts) |
| | Butylene glycol | -- | (in extracts) | -- | -- | -- | -- | -- | -- | -- |
| | Ectoine | 0.15 | 0.20 | 0.20 | 0.20 | 0.10 | 0.10 | 0.15 | 0.20 | 0.10 |
| | Erythritol | 0.15 | | | 0.20 | 0.10 | 0.15 | 0.15 | 0.30 | 0.20 |
| Vitamins | d-panthenol (pro-vitamin B5) | 0.10 | 0.05 | 0.10 | 0.20 | 0.15 | 0.15 | 0.05 | 0.05 | 0.20 |
| | d-Biotin (Vitamin B7) | -- | 0.05 | -- | -- | -- | -- | -- | -- | -- |
| | Niacinamide (Vitamin B3) | -- | 0.10 | 0.05 | 0 | -- | -- | -- | -- | -- |
| | Sodium ascorbyl phosphate (Vitamin C) | -- | 0.05 | 0.10 | -- | -- | -- | -- | -- | -- |
| Salts /Electrolytes | Sodium Chloride | 0.15 | -- | -- | 0.15 | 0.20 | 0.20 | 0.20 | 0.20 | 0.15 |
| | Potassium Chloride | 0.10 | 0.10 | 0.05 | 0.10 | 0.05 | 0.05 | 0.05 | 0.05 | 0.10 |
| | Sodium phosphate dibasic, anhydrous | 0.10 | 0.10 | 0.10 | 0.10 | 0.10 | 0.10 | 0.10 | 0.10 | 0.10 |
| | Sodium citrate, dihydrate | 0.10 | 0.10 | 0.10 | 0.10 | 0.10 | 0.10 | 0.10 | 0.10 | 0.10 |
| | Citric acid, monohydrate | 0.015 | 0.015 | 0.025 | 0.015 | 0.015 | 0.015 | 0.015 | 0.015 | 0.015 |
| Antimicrobial Agents | 1,2-hexanediol (and) caprylyl glycol | -- | 0.20 | -- | -- | -- | -- | -- | -- | -- |
| | Alexidine chloride, 0.5% solution | -- | 2.0 | 2.0 | -- | -- | -- | -- | -- | -- |
| Plant Extracts | Aloe Barbadensis Leaf Juice (concentrate 200:1) | 0.01 | -- | -- | 0.06 | 0.10 | 0.01 | 0.20 | 0.10 | 0.05 |
| | Licorice root (Glycyrrhiza glabra) extract in glycerin | 0.10 | -- | 0.10 | 0.01 | 0.15 | 0.10 | 0.15 | 0.20 | 0.15 |
| | Dandelion Root (taraxacum officinale) extract in glycerin | 0.05 | -- | -- | 0.18 | 0.02 | 0.20 | 0.05 | 0.15 | 0.10 |
| | Licorice root (Glycyrrhiza glabra) extract in butylene glycol | -- | 0.10 | -- | -- | -- | -- | -- | -- | -- |
| | White mulberry (Morus alba) bark extract in butylene glycol | -- | 0.10 | -- | -- | -- | -- | -- | -- | -- |
| | Cucumber Fruit (Cucumis Sative) Extract in glycerin | -- | -- | 0.10 | -- | -- | -- | -- | -- | -- |
| | Kelp (Ascophyllum Nodosum) Extract in butylene glycol | -- | 0.05 | 0.05 | -- | -- | -- | -- | -- | -- |
| | Eyebright (Euphrasia Officinalis) Extract in glycerin | -- | 0.05 | 0.05 | -- | -- | -- | -- | -- | -- |
| | L-menthol | -- | -- | 0.0025 | -- | -- | -- | -- | -- | -- |
| | Caffeine | -- | 0.02 | 0.02 | -- | -- | -- | -- | -- | -- |
| Non-ionic Surfactants /Micellar Cleansers | Sorbitan Oleate Decylglucoside Crosspolymer | 0.75 | 0.75 | 0.75 | 0.90 | 0.5 | 1.0 | 0.5 | 1.0 | 0.65 |
| | Decyl glucoside | 0.75 | 0.75 | 0.75 | 0.90 | 0.5 | 1.0 | 1.0 | 0.5 | 0.85 |

TABLE 1B

| Formulation component | Ingredient | Formulation (Wt%) | | | | | | | | |
|---|---|---|---|---|---|---|---|---|---|---|
| | | J | K | L | M | N | O | P | Q | R |
| Polymers /Viscosity-increasing Agent | Sodium Hyaluronate | 0.15 | 0.15 | 0.15 | 0.12 | 0.18 | 0.18 | 0.18 | 0.15 | 0.10 |
| Humectants | Propanediol | 0.80 | 0.60 | 1.05 | 0.80 | 0.60 | 0.80 | 1.05 | 0.75 | 0.55 |
| | Glycerin | (in extracts) | -- | (in extracts) | (in extracts) | (in extracts) | (in extracts) | (in extracts) | (in extracts) | (in extracts) |
| | Butylene glycol | -- | (in extracts) | -- | -- | -- | -- | -- | -- | -- |
| | Ectoine | 0.10 | 0.20 | 0.20 | 0.20 | 0.08 | 0.10 | 0.15 | 0.20 | 0.12 |
| | Erythritol | -- | -- | -- | -- | -- | -- | -- | -- | -- |
| Vitamins | d-panthenol (pro-vitamin B5) | 0.15 | 0.15 | 0.01 | 0.10 | 0.10 | 0.15 | 0.08 | 0.05 | 0.10 |
| | d-Biotin (Vitamin B7) | -- | 0.05 | -- | -- | -- | -- | -- | -- | -- |
| | Niacinamide (Vitamin B3) | 0.80 | 0.10 | 0.05 | 0.95 | 0.70 | 0.90 | 0.10 | 0.20 | 0.45 |
| | Sodium ascorbyl phosphate (Vitamin C) | 0.20 | 0.05 | 0.10 | 0.20 | 0.20 | 0.20 | 0.20 | 0.20 | 0.20 |
| Salts /Electrolytes | Sodium Chloride | -- | -- | -- | -- | -- | -- | -- | -- | -- |
| | Potassium Chloride | -- | 0.10 | 0.05 | -- | -- | -- | -- | -- | -- |
| | Sodium phosphate | -- | 0.10 | 0.10 | -- | -- | -- | -- | -- | -- |
| | dibasic, anhydrous | | | | | | | | | |
| | Sodium citrate, dihydrate | -- | 0.10 | 0.10 | -- | -- | -- | -- | -- | -- |
| | Citric acid, monohydrate | 0.025 | 0.015 | 0.025 | 0.025 | 0.025 | 0.025 | 0.025 | 0.025 | 0.025 |
| Antimicrobial Agents | 1,2-hexanediol (and) caprylyl glycol | -- | 0.20 | -- | -- | -- | -- | -- | -- | -- |
| | Alexidine chloride, 0.5% solution | 2.0 | 2.0 | 2.0 | 2.0 | 2.0 | 1.0 | 1.5 | 1.0 | 1.5 |
| Plant Extracts | Aloe Barbadensis Leaf Juice (concentrate 200:1) | -- | -- | -- | -- | -- | -- | -- | -- | -- |
| | Licorice root (Glycyrrhiza glabra) extract in glycerin | 0.15 | -- | 0.10 | 0.10 | 0.15 | 0.10 | 0.15 | 0.20 | 0.18 |
| | Dandelion Root (taraxacum officinale) extract in glycerin | -- | -- | -- | -- | -- | -- | -- | -- | -- |
| | Licorice root (Glycyrrhiza glabra) extract in butylene glycol | -- | 0.10 | -- | -- | -- | -- | -- | -- | -- |
| | White mulberry (Morus alba) bark extract in butylene glycol | -- | 0.10 | -- | -- | -- | -- | -- | -- | -- |
| | Cucumber Fruit (Cucumis Sative) Extract in glycerin | 0.20 | 0.15 | 0.10 | 0.10 | 0.05 | 0.15 | 0.02 | 0.18 | 0.10 |
| | Kelp (Ascophyllum Nodosum) Extract in butylene glycol | -- | -- | 0.20 | -- | -- | -- | -- | -- | -- |
| | Eyebright (Euphrasia Officinalis) Extract in glycerin | 0.05 | 0.01 | 0.10 | 0.05 | 0.20 | 0.10 | 0.18 | 0.18 | 0.01 |
| | L-menthol | -- | 0.02 | -- | -- | -- | -- | -- | -- | -- |
| | Caffeine | -- | -- | 0.02 | -- | -- | -- | -- | -- | --- |
| Non-ionic Surfactants /Micellar Cleansers | Sorbitan Oleate Decylglucoside Crosspolymer | 0.80 | 1.0 | 0.75 | 0.75 | 0.5 | 0.2 | 0.1 | 0.2 | 0.3 |
| | Decyl glucoside | 0.70 | 1.0 | 1.0 | 0.75 | 0.5 | 0.6 | 1.0 | 0.6 | 0.06 |

Formulations with the ingredients listed above can be prepared by mixing the recited ingredients, for example according to the proposed manufacturing process below:
1. Add water as 90% of batch weight to vessel.
2. Initiate mixing, for example at 700 rpm for lab mixer.
3. Disperse sodium hyaluronate into water.
4. Add pH-adjusting agents (e.g., add sodium citrate, sodium phosphate dibasic, citric acid, sodium ascorbyl phosphate).
5. Add (in any order, excluding optional ingredients as appropriate):
   a. Potassium chloride
   b. Ectoine
   c. Propanediol
   d. Menthol
   e. Panthenol
   f. Biotin
   g. Niacinamide
   h. Caffeine
   i. Licorice root extract
   j. Kelp extract
   k. Mulberry extract
   l. Eyebright extract
   m. Cucumber extract
   n. Preservative
6. Continue mixing for NLT 1 hour.
7. Reduce mixing speed, for example to 400 rpm for lab mixer.
8. Add decyl glucoside surfactant.
9. Add sugamulse D9 surfactant.
10. Check pH. If necessary, adjust pH with sodium hydroxide and/or hydrochloric acid to pH 7.5 +/- 0.1.
11. Add water to final batch weight.
12. (optional) Filter through 0.2-micron or 0.45-micron filter to reduce bioburden, if necessary.
13. If a sterile product is required, the product may be sterilized by gamma irradiation or autoclaving after packaging, or the product may be aseptically packaged after filtration by a 0.2-micron sterilizing filter.

The pH, osmolality, and color of the formulations are assessed.

Formulations listed above can be sterilized by autoclaving at 124° C. for 30 minutes. Once the autoclaved formulated solutions are cooled, the following steps are carried out:
1. Solution pH is measured to ensure that there is no more than 0.25 pH units change in pH.
2. Osmolality of the solution is measured to ensure that there is no more than 15 mOsm change in osmolality.
3. Color of the solution is evaluated to ensure that there is no visible darkening of the solution.

Autoclaved formulations not able to meet these criteria are deemed unstable and may be deemed unsuitable for use.

Formulations listed above can alternatively be sterilized by irradiating with gamma irradiation at 20 kGy - 40 kGy. Following irradiation, the following steps are carried out:
4. Solution pH is measured to ensure that there is no more than 0.25 pH units change in pH.
5. Osmolality of the solution is measured to ensure that there is no more than 15 mOsm, change in osmolality.
6. Color of the solution is evaluated to ensure that there is no visible darkening of the solution.

Irradiated formulations not able to meet these criteria are deemed unstable and may be deemed unsuitable for use

Batches of Formulation A were manufactured under representative conditions and sterilized at low-dose gamma irradiation (19-26 kGy) and high-dose gamma irradiation (40-50 kGy) and then stability was assessed.

TABLE 2

| Dose | | T=0 | 3 M/25° C. | 1 M/40° C. | 2 M/40° C. | 3 M/40° C. |
|---|---|---|---|---|---|---|
| 40-50 kGy | pH | 7.4 | 7.37 | 7.41 | 7.64 | 7.38 |
| | Osmolality | 322 | 323 | 340 | 282 | 322 |
| | Sterility | pass | n/a | n/a | n/a | n/a |
| | Weight Loss % | n/a | -0.20 | -0.20 | 0.00 | -0.14 |
| 19-26 kGy | pH | 7.5 | 7.36 | 7.31 | 7.91 | 7.36 |
| | Osmolality | 338 | 326 | 398 | 338 | 327 |
| | Sterility | pass | n/a | n/a | n/a | n/a |
| | Weight Loss | n/a | -0.20 | 3.00 | -0.07 | -0.16 |
| 19-26 kGy | pH | 7.6 | 7.43 | 7.43 | 7.68 | 7.40 |
| | Osmolality | 324 | 307 | 282 | 288 | 308 |
| | Sterility | pass | n/a | n/a | n/a | n/a |
| | Weight Loss | n/a | -0.17 | -0.20 | -0.05 | -0.19 |
| 19-26 kGy | pH | 7.4 | 6.98 | 7.08 | 7.11 | 6.99 |
| | Osmolality | 308 | 302 | 328 | 278 | 302 |
| | Sterility | pass | n/a | n/a | n/a | n/a |
| | Weight Loss | n/a | -0.18 | -1.20 | -0.14 | -0.18 |

What is claimed is:

1. A wipe for the eye area, comprising
a pad which is pre-moistened with a cleansing formulation, wherein the formulation comprises decyl glucoside and/or sorbitan oleate decylglucoside crosspolymer; and a package.

2. The wipe according to claim 1, wherein the formulation is sterilized by filtration, autoclaving, gamma irradiation and/or electron-beam.

3. The wipe according to claim 1, wherein the formulation is free of parabens, thiazolinones, and formaldehyde-donor preservatives.

4. The wipe according to claim 1, wherein the formulation is free of polyethylene glycol (PEG) and PEG-containing ingredients.

5. The wipe according to claim 1, wherein the formulation further comprises at least one natural humectant.

6. The wipe according to claim 5, wherein the at least one natural humectant is chosen from sodium hyaluronate, propanediol, erythritol, and ectoine.

7. The wipe according to claim 1, wherein the formulation further comprises a citrate-phosphate buffer providing the formulation with a pH of 7 - 8.

8. The wipe according to claim 1, wherein the formulation further comprises at least one plant extract.

9. The wipe according to claim 8, wherein the at least one plant extract is chosen from aloe barbadensis leaf juice, licorice root (glycyrrhiza glabra) extract, dandelion root (taraxacum officinale) extract, kelp (ascophyllum nodosum) extract, cucumber fruit (cucumis sativa) extract, eyebright (euphrasia officinalis) extract, caffeine, menthol, and white mulberry (morus alba) bark extract.

10. The wipe according to claim 1, wherein the formulation further comprises at least one vitamin.

11. The wipe according to claim 10, wherein the at least one vitamin is chosen from d-panthenol (pro-vitamin B), tocopherol (vitamin E), sodium ascorbyl phosphate (vitamin C), niacinamide (vitamin B3), d-panthenol (pro-vitamin B5), and d-biotin (vitamin B7).

12. The wipe according to claim 1, wherein the formulation further comprises at least one antimicrobial agent.

13. The wipe according to claim 12, wherein the at least one antimicrobial agent is chosen from caprylyl glycol, phenylpropanol, 1,2-hexanediol, caprylyl glycol, and alexidine chloride.

14. The wipe according to claim 1, wherein the formulation comprises:
0.10 wt% to 0.24 wt% sodium hyaluronate;
0.55 wt% to 1.05 wt% propanediol;
0.08 wt% to 0.20 wt% ectoine;
0.01 wt% to 0.20 wt% d-panthenol (pro-vitamin B5);
0.015 wt% to 0.025 wt% citric acid monohydrate;
0.10 wt% to 1.0 wt% sorbitan oleate decylglucoside crosspolymer; and
0.06 wt% to 1.0 wt% decyl glucoside.

15. The wipe according to claim 14, wherein the formulation further comprises:
0.01 wt% to 0.30 wt% erythritol;
0.01 wt% to 0.05 wt% d-biotin (vitamin B7);
0.01 wt% 0.95 wt% niacinamide (vitamin B3);
0.01 wt% to 0.20 wt% sodium ascorbyl phosphate (vitamin C);
0.01 wt% to 0.20 wt% sodium chloride;
0.01 wt% to 0.10 wt% potassium chloride;
0.01 wt% to 0.10 wt% sodium phosphate dibasic;
0.01 wt% to 0.10 wt% sodium citrate dihydrate;
0.01 wt% to 0.20 wt% 1,2-hexanediol and caprylyl glycol;
0.01 wt% to 2.0 wt% alexidine chloride, 0.5% solution;
0.01 wt% to 0.20 wt% aloe barbadensis leaf juice (concentrate 200:1);
0.01 wt% to 0.20 wt% licorice root (glycyrrhiza glabra) extract in glycerin or butylene glycol;
0.01 wt% to 0.20 wt% dandelion root (taraxacum officinale) extract in glycerin;
0.01 wt% to 0.10 wt% white mulberry (morus alba) bark extract in butylene glycol;
0.01 wt% to 0.20 wt% cucumber fruit (cucumis sative) extract in glycerin;
0.01 wt% to 0.20 wt% kelp (ascophyllum nodosum) extract in butylene glycol;
0.01 wt% to 0.20 wt% eyebright (euphrasia officinalis) extract in glycerin;
0.01 wt% to 0.02 wt% caffeine; and/or
0.01 wt% to 0.02 wt% L-menthol.

16. The wipe according to claim 1, wherein the formulation comprises:
0.12 wt% sodium hyaluronate;
0.80 wt% propanediol;
0.15 wt% ectoine;
0.15 wt% erythritol;
0.10 wt% d-panthenol (pro-vitamin B5);
0.15 wt% sodium chloride;
0.10 wt% potassium chloride;
0.10 wt% sodium phosphate dibasic;
0.10 wt% sodium citrate dihydrate;
0.015 wt% citric acid monohydrate;
0.01 wt% aloe barbadensis leaf juice (concentrate 200:1);
0.10 wt% licorice root (glycyrrhiza glabra) extract in glycerin;
0.05 wt% dandelion root (taraxacum officinale) extract in glycerin;
0.75 wt% sorbitan oleate decylglucoside crosspolymer; and
0.75 wt% decyl glucoside.

17. The wipe according to claim 1, wherein the formulation comprises:
0.12 wt% sodium hyaluronate;
0.80 wt% propanediol;
0.20 wt% ectoine;
0.05 wt% d-panthenol (pro-vitamin B5);
0.05 wt% d-biotin (vitamin B7);
0.10 wt% niacinamide (vitamin B3);
0.05 wt% sodium ascorbyl phosphate (vitamin C);
0.10 wt% potassium chloride;
0.10 wt% sodium phosphate dibasic;
0.10 wt% sodium citrate dihydrate;
0.015 wt% citric acid monohydrate;
0.20 wt% 1,2-hexanediol and caprylyl glycol;
2.0 wt% alexidine chloride, 0.5% solution;
0.10 wt% licorice root (glycyrrhiza glabra) extract in butylene glycol;
0.10 wt% white mulberry (morus alba) bark extract in butylene glycol;
0.05 wt% kelp (ascophyllum nodosum) extract in butylene glycol;
0.05 wt% eyebright (euphrasia officinalis) extract in glycerin;
0.02 wt% caffeine;
0.75 wt% sorbitan oleate decylglucoside crosspolymer; and
0.75 wt% decyl glucoside.

18. The wipe according to claim 1, wherein the formulation comprises:
0.12 wt% sodium hyaluronate;
0.80 wt% propanediol;
0.20 wt% ectoine;
0.10 wt% d-panthenol (pro-vitamin B5);
0.05 wt% niacinamide (vitamin B3);
0.10 wt% sodium ascorbyl phosphate (vitamin C);
0.05 wt% potassium chloride;
0.10 wt% sodium phosphate dibasic;
0.10 wt% sodium citrate dihydrate;
0.025 wt% citric acid monohydrate;
2.0 wt% alexidine chloride, 0.5% solution;
0.10 wt% licorice root (glycyrrhiza glabra) extract in glycerin;
0.10 wt% cucumber fruit (cucumis sative) extract in glycerin;
0.05 wt% kelp (ascophyllum nodosum) extract in butylene glycol;
0.05 wt% eyebright (euphrasia officinalis) extract in glycerin;
0.0025 wt% L-menthol;
0.02 wt% caffeine;
0.75 wt% sorbitan oleate decylglucoside crosspolymer; and
0.75 wt% decyl glucoside.

19. The wipe according to claim 1, wherein the formulation comprises:
0.12 wt% sodium hyaluronate;
0.80 wt% propanediol;
0.20 wt% ectoine;
0.20 wt% erythritol;
0.20 wt% d-panthenol (pro-vitamin B5);
0.15 wt% sodium chloride;
0.10 wt% potassium chloride;
0.10 wt% sodium phosphate dibasic;
0.10 wt% sodium citrate dihydrate;
0.015 wt% citric acid monohydrate;
0.06 wt% aloe barcadensis leaf juice (concentrate 200:1);
0.01 wt% licorice root (glycyrrhiza glabra) extract in glycerin;
0.18 wt% dandelion root (taraxacum officinale) extract in glycerin;
0.90 wt% sorbitan oleate decylglucoside crosspolymer; and
0.90 wt% decyl glucoside.

20. The wipe according to claim 1, wherein the formulation comprises:
0.18 wt% sodium hyaluronate;
0.60 wt% propanediol;
0.10 wt% ectoine;
0.10 wt% erythritol;
0.15 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.10 wt% aloe barcadensis leaf juice (concentrate 200:1);
0.15 wt% licorice root (glycyrrhiza glabra) extract in glycerin;
0.02 wt% dandelion root (taraxacum officinale) extract in glycerin;
0.5 wt% sorbitan oleate decylglucoside crosspolymer; and
0.5 wt% decyl glucoside.

21. The wipe according to claim 1, wherein the formulation comprises:
0.24 wt% sodium hyaluronate;
0.80 wt% propanediol;
0.10 wt% ectoine;
0.15 wt% erythritol;
0.15 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.01 wt% aloe barcadensis leaf juice (concentrate 200:1);
0.10 wt% licorice root (glycyrrhiza glabra) extract in glycerin;
0.20 wt% dandelion root (taraxacum officinale) extract in glycerin;
1.0 wt% sorbitan oleate decylglucoside crosspolymer; and
1.0 wt% decyl glucoside.

22. The wipe according to claim 1, wherein the formulation comprises:
0.24 wt% sodium hyaluronate;
1.05 wt% propanediol;
0.15 wt% ectoine;
0.15 wt% erythritol;
0.05 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.20 wt% aloe barcadensis leaf juice (concentrate 200:1);
0.15 wt% licorice root (glycyrrhiza glabra) extract in glycerin;
0.05 wt% dandelion root (taraxacum officinale) extract in glycerin;
0.5 wt% sorbitan oleate decylglucoside crosspolymer; and
1.0 wt% decyl glucoside.

23. The wipe according to claim 1, wherein the formulation comprises:
0.15 wt% sodium hyaluronate;
0.75 wt% propanediol;
0.20 wt% ectoine;
0.30 wt% erythritol;
0.05 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.10 wt% aloe barcadensis leaf juice (concentrate 200:1);
0.20 wt% licorice root (glycyrrhiza glabra) extract in glycerin;
0.15 wt% dandelion root (taraxacum officinale) extract in glycerin;
1.0 wt% sorbitan oleate decylglucoside crosspolymer; and
0.5 wt% decyl glucoside.

24. The wipe according to claim 1, wherein the formulation comprises:
0.15 wt% sodium hyaluronate;
0.75 wt% propanediol;
0.10 wt% ectoine;
0.20 wt% erythritol;
0.20 wt% d-panthenol (pro-vitamin B5);
0.15 wt% sodium chloride;
0.10 wt% potassium chloride;
0.10 wt% sodium phosphate dibasic;
0.10 wt% sodium citrate dihydrate;
0.015 wt% citric acid monohydrate;
0.05 wt% aloe barcadensis leaf juice (concentrate 200:1);
0.15 wt% licorice root (glycyrrhiza glabra) extract in glycerin;
0.10 wt% dandelion root (taraxacum officinale) extract in glycerin;
0.65 wt% sorbitan oleate decylglucoside crosspolymer; and
0.85 wt% decyl glucoside.

25. The wipe according to claim 1, wherein the formulation comprises:
0.15 wt% sodium hyaluronate;
0.80 wt% propanediol;
0.10 wt% ectoine;
0.15 wt% d-panthenol (pro-vitamin B5);
0.80 wt% niacinamide (vitamin B3);
0.20 wt% sodium ascorbyl phosphate (vitamin C);
0.025 wt% citric acid monohydrate;
2.0 wt% alexidine chloride, 0.5% solution;
0.15 wt% licorice root (glycyrrhiza glabra) extract in glycerin;
0.20 wt% cucumber fruit (cucumis sative) extract in glycerin;
0.05 wt% eyebright (euphrasia officinalis) extract in glycerin;
0.80 wt% sorbitan oleate decylglucoside crosspolymer; and
0.70 wt% decyl glucoside.

26. The wipe according to claim 1, wherein the formulation comprises:
0.15 wt% sodium hyaluronate;
0.60 wt% propanediol;
0.20 wt% ectoine;
0.15 wt% d-panthenol (pro-vitamin B5);
0.05 wt% d-biotin (vitamin B7);
0.10 wt% niacinamide (vitamin B3);
0.05 wt% sodium ascorbyl phosphate (vitamin C);
0.10 wt% potassium chloride;
0.10 wt% sodium phosphate dibasic;
0.10 wt% sodium citrate dihydrate;
0.015 wt% citric acid monohydrate;
0.20 wt% 1,2-hexanediol and caprylyl glycol;
2.0 wt% alexidine chloride, 0.5% solution;
0.10 wt% licorice root (glycyrrhiza glabra) extract in butylene glycol;
0.10 wt% white mulberry (morus alba) bark extract in butylene glycol;
0.15 wt% cucumber fruit (cucumis sative) extract in glycerin;
0.01 wt% eyebright (euphrasia officinalis) extract in glycerin;
0.02 wt% L-menthol;
1.0 wt% sorbitan oleate decylglucoside crosspolymer; and
1.0 wt% decyl glucoside.

27. The wipe according to claim 1, wherein the formulation comprises:
0.15 wt% sodium hyaluronate;
1.05 wt% propanediol;
0.20 wt% ectoine;
0.01 wt% d-panthenol (pro-vitamin B5);
0.05 wt% niacinamide (vitamin B3);
0.10 wt% sodium ascorbyl phosphate (vitamin C);
0.05 wt% potassium chloride;
0.10 wt% sodium phosphate dibasic;
0.10 wt% sodium citrate dihydrate;
0.025 wt% citric acid monohydrate;
2.0 wt% alexidine chloride, 0.5% solution;
0.10 wt% licorice root (glycyrrhiza glabra) extract in glycerin;
0.10 wt% cucumber fruit (cucumis sative) extract in glycerin;
0.20 wt% kelp (ascophyllum nodosum) extract in butylene glycol;
0.10 wt% eyebright (euphrasia officinalis) extract in glycerin;
0.02 wt% caffeine;
0.75 wt% sorbitan oleate decylglucoside crosspolymer; and
1.0 wt% decyl glucoside.

28. The wipe according to claim 1, wherein the formulation comprises:
0.12 wt% sodium hyaluronate;
0.80 wt% propanediol;
0.20 wt% ectoine;
0.10 wt% d-panthenol (pro-vitamin B5);
0.95 wt% niacinamide (vitamin B3);
0.20 wt% sodium ascorbyl phosphate (vitamin C);
0.025 wt% citric acid monohydrate;
2.0 wt% alexidine chloride, 0.5% solution;
0.10 wt% licorice root (glycyrrhiza glabra) extract in glycerin;
0.10 wt% cucumber fruit (cucumis sative) extract in glycerin;
0.05 wt% eyebright (euphrasia officinalis) extract in glycerin;
0.75 wt% sorbitan oleate decylglucoside crosspolymer; and
0.75 wt% decyl glucoside.

29. The wipe according to claim 1, wherein the formulation comprises:
0.18 wt% sodium hyaluronate;
0.60 wt% propanediol;
0.08 wt% ectoine;
0.10 wt% d-panthenol (pro-vitamin B5);
0.70 wt% niacinamide (vitamin B3);
0.20 wt% sodium ascorbyl phosphate (vitamin C);
0.025 wt% citric acid monohydrate;
2.0 wt% alexidine chloride, 0.5% solution;
0.15 wt% licorice root (glycyrrhiza glabra) extract in glycerin;
0.05 wt% cucumber fruit (cucumis sative) extract in glycerin;
0.20 wt% eyebright (euphrasia officinalis) extract in glycerin;
0.5 wt% sorbitan oleate decylglucoside crosspolymer; and
0.5 wt% decyl glucoside.

30. The wipe according to claim 1, wherein the formulation comprises:
0.18 wt% sodium hyaluronate;
0.80 wt% propanediol;
0.10 wt% ectoine;
0.15 wt% d-panthenol (pro-vitamin B5);
0.90 wt% niacinamide (vitamin B3);
0.20 wt% sodium ascorbyl phosphate (vitamin C);
0.025 wt% citric acid monohydrate;
1.0 wt% alexidine chloride, 0.5% solution;
0.10 wt% licorice root (glycyrrhiza glabra) extract in glycerin;
0.15 wt% cucumber fruit (cucumis sative) extract in glycerin;
0.10 wt% eyebright (euphrasia officinalis) extract in glycerin;
0.2 wt% sorbitan oleate decylglucoside crosspolymer; and
0.6 wt% decyl glucoside.

31. The wipe according to claim 1, wherein the formulation comprises:
0.18 wt% sodium hyaluronate;
1.05 wt% propanediol;
0.15 wt% ectoine;
0.08 wt% d-panthenol (pro-vitamin B5);
0.10 wt% niacinamide (vitamin B3);
0.20 wt% sodium ascorbyl phosphate (vitamin C);
0.025 wt% citric acid monohydrate;
1.5 wt% alexidine chloride, 0.5% solution;
0.15 wt% licorice root (glycyrrhiza glabra) extract in glycerin;
0.02 wt% cucumber fruit (cucumis sative) extract in glycerin;
0.18 wt% eyebright (euphrasia officinalis) extract in glycerin;
0.1 wt% sorbitan oleate decylglucoside crosspolymer; and
1.0 wt% decyl glucoside.

32. The wipe according to claim 1, wherein the formulation comprises:
0.1 wt% sodium hyaluronate;
0.75 wt% propanediol;
0.20 wt% ectoine;
0.05 wt% d-panthenol (pro-vitamin B5);
0.20 wt% niacinamide (vitamin B3);
0.20 wt% sodium ascorbyl phosphate (vitamin C);
0.025 wt% citric acid monohydrate;
1.0 wt% alexidine chloride, 0.5% solution;
0.20 wt% licorice root (glycyrrhiza glabra) extract in glycerin;
0.18 wt% cucumber fruit (cucumis sative) extract in glycerin;
0.18 wt% eyebright (euphrasia officinalis) extract in glycerin;
0.2 wt% sorbitan oleate decylglucoside crosspolymer; and
0.6 wt% decyl glucoside.

33. The wipe according to claim 1, wherein the formulation comprises:
0.10 wt% sodium hyaluronate;
0.55 wt% propanediol;
0.12 wt% ectoine;
0.10 wt% d-panthenol (pro-vitamin B5);
0.45 wt% niacinamide (vitamin B3);
0.20 wt% sodium ascorbyl phosphate (vitamin C);
0.025 wt% citric acid monohydrate;
1.5 wt% alexidine chloride, 0.5% solution;
0.18 wt% licorice root (glycyrrhiza glabra) extract in glycerin;
0.10 wt% cucumber fruit (cucumis sative) extract in glycerin;
0.01 wt% eyebright (euphrasia officinalis) extract in glycerin;
0.3 wt% sorbitan oleate decylglucoside crosspolymer; and
0.06 wt% decyl glucoside.

34. A formulation for cleansing the eye area comprising decyl glucoside and sorbitan oleate decylglucoside crosspolymer.

35. The formulation according to claim 34, which is compatible with sterilization by filtration, autoclaving, gamma irradiation and/or electron-beam.

36. The formulation according to claim 34, which is stable.

37. The formulation according to claim 34, which is free of parabens, thiazolinones, and formaldehyde-donor preservatives.

38. The formulation according to claim 34, which is free of polyethylene glycol (PEG) and PEG-containing ingredients.

39. The formulation according to claim 34, further comprising at least one natural humectant.

40. The formulation according to claim 39, wherein the at least one natural humectant is chosen from sodium hyaluronate, propanediol, erythritol, and ectoine.

41. The formulation according to claim 34, further comprising sodium hyaluronate, propanediol, erythritol, and ectoine.

42. The formulation according to claim 34, further comprising sodium hyaluronate and at least one natural humectant having antioxidant activity.

43. The formulation according to claim 42, wherein the at least one natural humectant having antioxidant activity is chosen from erythritol and ectoine.

44. The formulation according to claim 43, further comprising propanediol.

45. The formulation according to claim 34, further comprising at least one buffer.

46. The formulation according to claim 45, wherein the at least one buffer is chosen from citrate-phosphate buffers.

47. The formulation according to claim 45 46, wherein the at least one buffer provides the formulation with a pH of 6 - 8.

48. The formulation according to claim 34, further comprising at least one plant extract.

49. The formulation according to claim 48, wherein the at least one plant extract is chosen from aloe barbadensis leaf juice, licorice root (glycyrrhiza glabra) extract, dandelion root (taraxacum officinale) extract, cucumber fruit (cucumber sative) extract, kelp (ascophyllum nodosum) extract, eyebright (euphrasia officinalis) extract, caffeine, menthol, and white mulberry (morus alba) bark extract.

50. The formulation according to claim 34, further comprising at least one vitamin.

51. The formulation according to claim 50, wherein the at least one vitamin is chosen from d-panthenol (pro-vitamin B), tocopherol (vitamin E), sodium ascorbyl phosphate (vitamin C), niacinamide (vitamin B3), d-panthenol (pro-vitamin B5), and d-biotin (vitamin B7).

52. The formulation according to claim 34, further comprising phenylethyl resorcinol.

53. The formulation according to claim 34, further comprising at least one antimicrobial agent.

54. The formulation according to claim 53, wherein the at least one antimicrobial agent is chosen from caprylyl glycol, phenylpropanol, 1,2-hexanediol, caprylyl glycol, and alexidine chloride.

55. The formulation according to claim 34, comprising:
0.10 wt% to 0.24 wt% sodium hyaluronate;
0.55 wt% to 1.05 wt% propanediol;
0.08 wt% to 0.20 wt% ectoine;
0.01 wt% to 0.20 wt% d-panthenol (pro-vitamin B5);
0.015 wt% to 0.025 wt% citric acid monohydrate;
0.10 wt% to 1.0 wt% sorbitan oleate decylglucoside crosspolymer; and
0.06 wt% to 1.0 wt% decyl glucoside.

56. The formulation according to claim 55, further comprising:
0.01 wt% to 0.30 wt% erythritol;
0.01 wt% to 0.05 wt% d-biotin (vitamin B7);
0.01 wt% 0.95 wt% niacinamide (vitamin B3);
0.01 wt% to 0.20 wt% sodium ascorbyl phosphate (vitamin C);
0.01 wt% to 0.20 wt% sodium chloride;
0.01 wt% to 0.10 wt% potassium chloride;
0.01 wt% to 0.10 wt% sodium phosphate dibasic;
0.01 wt% to 0.10 wt% sodium citrate dihydrate;
0.01 wt% to 0.20 wt% 1,2-hexanediol and caprylyl glycol;
0.01 wt% to 2.0 wt% alexidine chloride, 0.5% solution;
0.01 wt% to 0.20 wt% aloe barbadensis leaf juice (concentrate 200:1);
0.01 wt% to 0.20 wt% licorice root (glycyrrhiza glabra) extract in glycerin or butylene glycol;
0.01 wt% to 0.20 wt% dandelion roon (taraxacum officinale) extract in glycerin;
0.01 wt% to 0.10 wt% white mulberry (morus alba) bark extract in butylene glycol;
0.01 wt% to 0.20 wt% cucumber fruit (cucumis sative) extract in glycerin;
0.01 wt% to 0.20 wt% kelp (ascophyllum nodosum) extract in butylene glycol;
0.01 wt% to 0.20 wt% eyebright (euphrasia officinalis) extract in glycerin;
0.01 wt% to 0.02 wt% caffeine; and/or
0.01 wt% to 0.02 wt% L-menthol.

57. The formulation according to claim 34, comprising:
0.12 wt% sodium hyaluronate;
0.80 wt% propanediol;
0.15 wt% ectoine;
0.15 wt% erythritol;
0.10 wt% d-panthenol (pro-vitamin B5);
0.15 wt% sodium chloride;
0.10 wt% potassium chloride;
0.10 wt% sodium phosphate dibasic;
0.10 wt% sodium citrate dihydrate;
0.015 wt% citric acid monohydrate;
0.01 wt% aloe barbadensis leaf juice (concentrate 200:1);
0.10 wt% licorice root (glycyrrhiza glabra) extract in glycerin;
0.05 wt% dandelion roon (taraxacum officinale) extract in glycerin;
0.75 wt% sorbitan oleate decylglucoside crosspolymer; and
0.75 wt% decyl glucoside.

58. The formulation according to claim 34, comprising:
0.12 wt% sodium hyaluronate;
0.80 wt% propanediol;
0.20 wt% ectoine;
0.05 wt% d-panthenol (pro-vitamin B5);
0.05 wt% d-biotin (vitamin B7);
0.10 wt% niacinamide (vitamin B3);
0.05 wt% sodium ascorbyl phosphate (vitamin C);
0.10 wt% potassium chloride;
0.10 wt% sodium phosphate dibasic;
0.10 wt% sodium citrate dihydrate;
0.015 wt% citric acid monohydrate;
0.20 wt% 1,2-hexanediol and caprylyl glycol;
2.0 wt% alexidine chloride, 0.5% solution;
0.10 wt% licorice root (glycyrrhiza glabra) extract in butylene glycol;
0.10 wt% white mulberry (morus alba) bark extract in butylene glycol;
0.05 wt% kelp (ascophyllum nodosum) extract in butylene glycol;
0.05 wt% eyebright (euphrasia officinalis) extract in glycerin;
0.02 wt% caffeine;
0.75 wt% sorbitan oleate decylglucoside crosspolymer; and
0.75 wt% decyl glucoside.

59. The formulation according to claim 34, comprising:
0.12 wt% sodium hyaluronate;
0.80 wt% propanediol;
0.20 wt% ectoine;
0.10 wt% d-panthenol (pro-vitamin B5);
0.05 wt% niacinamide (vitamin B3);
0.10 wt% sodium ascorbyl phosphate (vitamin C);
0.05 wt% potassium chloride;
0.10 wt% sodium phosphate dibasic;
0.10 wt% sodium citrate dihydrate;
0.025 wt% citric acid monohydrate;
2.0 wt% alexidine chloride, 0.5% solution;
0.10 wt% licorice root (glycyrrhiza glabra) extract in glycerin;
0.10 wt% cucumber fruit (cucumis sative) extract in glycerin;
0.05 wt% kelp (ascophyllum nodosum) extract in butylene glycol;
0.05 wt% eyebright (euphrasia officinalis) extract in glycerin;
0.0025 wt% L-menthol;
0.02 wt% caffeine;
0.75 wt% sorbitan oleate decylglucoside crosspolymer; and
0.75 wt% decyl glucoside.

60. The formulation according to claim 34, comprising:
0.12 wt% sodium hyaluronate;
0.80 wt% propanediol;
0.20 wt% ectoine;
0.20 wt% erythritol;
0.20 wt% d-panthenol (pro-vitamin B5);
0.15 wt% sodium chloride;
0.10 wt% potassium chloride;
0.10 wt% sodium phosphate dibasic;
0.10 wt% sodium citrate dihydrate;
0.015 wt% citric acid monohydrate;
0.06 wt% aloe barcadensis leaf juice (concentrate 200:1);
0.01 wt% licorice root (glycyrrhiza glabra) extract in glycerin;
0.18 wt% dandelion root (taraxacum officinale) extract in glycerin;
0.90 wt% sorbitan oleate decylglucoside crosspolymer; and
0.90 wt% decyl glucoside.

61. The formulation according to claim 34, comprising:
0.18 wt% sodium hyaluronate;
0.60 wt% propanediol;
0.10 wt% ectoine;
0.10 wt% erythritol;
0.15 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.10 wt% aloe barcadensis leaf juice (concentrate 200:1);
0.15 wt% licorice root (glycyrrhiza glabra) extract in glycerin;
0.02 wt% dandelion root (taraxacum officinale) extract in glycerin;
0.5 wt% sorbitan oleate decylglucoside crosspolymer; and
0.5 wt% decyl glucoside.

62. The formulation according to claim 34, comprising:
0.24 wt% sodium hyaluronate;
0.80 wt% propanediol;
0.10 wt% ectoine;
0.15 wt% erythritol;
0.15 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.01 wt% aloe barcadensis leaf juice (concentrate 200:1);
0.10 wt% licorice root (glycyrrhiza glabra) extract in glycerin;
0.20 wt% dandelion root (taraxacum officinale) extract in glycerin;
1.0 wt% sorbitan oleate decylglucoside crosspolymer; and
1.0 wt% decyl glucoside.

63. The formulation according to claim 34, comprising:
0.24 wt% sodium hyaluronate;
1.05 wt% propanediol;
0.15 wt% ectoine;
0.15 wt% erythritol;
0.05 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.20 wt% aloe barcadensis leaf juice (concentrate 200:1);
0.15 wt% licorice root (glycyrrhiza glabra) extract in glycerin;
0.05 wt% dandelion root (taraxacum officinale) extract in glycerin;
0.5 wt% sorbitan oleate decylglucoside crosspolymer; and
1.0 wt% decyl glucoside.

64. The formulation according to claim 34, comprising:
0.15 wt% sodium hyaluronate;
0.75 wt% propanediol;
0.20 wt% ectoine;
0.30 wt% erythritol;
0.05 wt% d-panthenol (pro-vitamin B5);
0.20 wt% sodium chloride;
0.05 wt% potassium chloride;
0.10 wt% sodium phosphate dibasic;
0.10 wt% sodium citrate dihydrate;
0.015 wt% citric acid monohydrate;
0.10 wt% aloe barcadensis leaf juice (concentrate 200:1);
0.20 wt% licorice root (glycyrrhiza glabra) extract in glycerin;
0.15 wt% dandelion root (taraxacum officinale) extract in glycerin;
1.0 wt% sorbitan oleate decylglucoside crosspolymer; and
0.5 wt% decyl glucoside.

65. The formulation according to claim 34, comprising:
0.15 wt% sodium hyaluronate;
0.75 wt% propanediol;
0.10 wt% ectoine;
0.20 wt% erythritol;
0.20 wt% d-panthenol (pro-vitamin B5);
0.15 wt% sodium chloride;
0.10 wt% potassium chloride;
0.10 wt% sodium phosphate dibasic;
0.10 wt% sodium citrate dihydrate;
0.015 wt% citric acid monohydrate;
0.05 wt% aloe barcadensis leaf juice (concentrate 200:1);
0.15 wt% licorice root (glycyrrhiza glabra) extract in glycerin;
0.10 wt% dandelion root (taraxacum officinale) extract in glycerin;
0.65 wt% sorbitan oleate decylglucoside crosspolymer; and
0.85 wt% decyl glucoside.

66. The formulation according to claim 34, comprising:
0.15 wt% sodium hyaluronate;
0.80 wt% propanediol;
0.10 wt% ectoine;
0.15 wt% d-panthenol (pro-vitamin B5);
0.80 wt% niacinamide (vitamin B3);
0.20 wt% sodium ascorbyl phosphate (vitamin C);
0.025 wt% citric acid monohydrate;
2.0 wt% alexidine chloride, 0.5% solution;
0.15 wt% licorice root (glycyrrhiza glabra) extract in glycerin;
0.20 wt% cucumber fruit (cucumis sative) extract in glycerin;
0.05 wt% eyebright (euphrasia officinalis) extract in glycerin;
0.80 wt% sorbitan oleate decylglucoside crosspolymer; and
0.70 wt% decyl glucoside.

67. The formulation according to claim 34, comprising:
0.15 wt% sodium hyaluronate;
0.60 wt% propanediol;
0.20 wt% ectoine;
0.15 wt% d-panthenol (pro-vitamin B5);
0.05 wt% d-biotin (vitamin B7);
0.10 wt% niacinamide (vitamin B3);
0.05 wt% sodium ascorbyl phosphate (vitamin C);
0.10 wt% potassium chloride;
0.10 wt% sodium phosphate dibasic;
0.10 wt% sodium citrate dihydrate;
0.015 wt% citric acid monohydrate;
0.20 wt% 1,2-hexanediol and caprylyl glycol;
2.0 wt% alexidine chloride, 0.5% solution;
0.10 wt% licorice root (glycyrrhiza glabra) extract in butylene glycol;
0.10 wt% white mulberry (morus alba) bark extract in butylene glycol;
0.15 wt% cucumber fruit (cucumis sative) extract in glycerin;
0.01 wt% eyebright (euphrasia officinalis) extract in glycerin;
0.02 wt% L-menthol;
1.0 wt% sorbitan oleate decylglucoside crosspolymer; and
1.0 wt% decyl glucoside.

68. The formulation according to claim 34, comprising:
0.15 wt% sodium hyaluronate;
1.05 wt% propanediol;
0.20 wt% ectoine;
0.01 wt% d-panthenol (pro-vitamin B5);
0.05 wt% niacinamide (vitamin B3);
0.10 wt% sodium ascorbyl phosphate (vitamin C);
0.05 wt% potassium chloride;
0.10 wt% sodium phosphate dibasic;
0.10 wt% sodium citrate dihydrate;
0.025 wt% citric acid monohydrate;
2.0 wt% alexidine chloride, 0.5% solution;
0.10 wt% licorice root (glycyrrhiza glabra) extract in glycerin;
0.10 wt% cucumber fruit (cucumis sative) extract in glycerin;
0.20 wt% kelp (ascophyllum nodosum) extract in butylene glycol;
0.10 wt% eyebright (euphrasia officinalis) extract in glycerin;
0.02 wt% caffeine;
0.75 wt% sorbitan oleate decylglucoside crosspolymer; and
1.0 wt% decyl glucoside.

69. The formulation according to claim 34, comprising:
0.12 wt% sodium hyaluronate;
0.80 wt% propanediol;
0.20 wt% ectoine;
0.10 wt% d-panthenol (pro-vitamin B5);
0.95 wt% niacinamide (vitamin B3);
0.20 wt% sodium ascorbyl phosphate (vitamin C);
0.025 wt% citric acid monohydrate;
2.0 wt% alexidine chloride, 0.5% solution;
0.10 wt% licorice root (glycyrrhiza glabra) extract in glycerin;
0.10 wt% cucumber fruit (cucumis sative) extract in glycerin;
0.05 wt% eyebright (euphrasia officinalis) extract in glycerin;
0.75 wt% sorbitan oleate decylglucoside crosspolymer; and
0.75 wt% decyl glucoside.

70. The formulation according to claim 34, comprising:
0.18 wt% sodium hyaluronate;
0.60 wt% propanediol;
0.08 wt% ectoine;
0.10 wt% d-panthenol (pro-vitamin B5);
0.70 wt% niacinamide (vitamin B3);
0.20 wt% sodium ascorbyl phosphate (vitamin C);
0.025 wt% citric acid monohydrate;
2.0 wt% alexidine chloride, 0.5% solution;
0.15 wt% licorice root (glycyrrhiza glabra) extract in glycerin;
0.05 wt% cucumber fruit (cucumis sative) extract in glycerin;
0.20 wt% eyebright (euphrasia officinalis) extract in glycerin;
0.5 wt% sorbitan oleate decylglucoside crosspolymer; and
0.5 wt% decyl glucoside.

71. The formulation according to claim 34, comprising:
0.18 wt% sodium hyaluronate;
0.80 wt% propanediol;
0.10 wt% ectoine;
0.15 wt% d-panthenol (pro-vitamin B5);
0.90 wt% niacinamide (vitamin B3);
0.20 wt% sodium ascorbyl phosphate (vitamin C);
0.025 wt% citric acid monohydrate;
1.0 wt% alexidine chloride, 0.5% solution;
0.10 wt% licorice root (glycyrrhiza glabra) extract in glycerin;
0.15 wt% cucumber fruit (cucumis sative) extract in glycerin;
0.10 wt% eyebright (euphrasia officinalis) extract in glycerin;
0.2 wt% sorbitan oleate decylglucoside crosspolymer; and
0.6 wt% decyl glucoside.

72. The formulation according to claim 34, comprising:
0.18 wt% sodium hyaluronate;
1.05 wt% propanediol;
0.15 wt% ectoine;
0.08 wt% d-panthenol (pro-vitamin B5);
0.10 wt% niacinamide (vitamin B3);
0.20 wt% sodium ascorbyl phosphate (vitamin C);
0.025 wt% citric acid monohydrate;
1.5 wt% alexidine chloride, 0.5% solution;
0.15 wt% licorice root (glycyrrhiza glabra) extract in glycerin;
0.02 wt% cucumber fruit (cucumis sative) extract in glycerin;
0.18 wt% eyebright (euphrasia officinalis) extract in glycerin;
0.1 wt% sorbitan oleate decylglucoside crosspolymer; and
1.0 wt% decyl glucoside.

73. The formulation according to claim 34, comprising:
0.1 wt% sodium hyaluronate;
0.75 wt% propanediol;
0.20 wt% ectoine;
0.05 wt% d-panthenol (pro-vitamin B5);
0.20 wt% niacinamide (vitamin B3);
0.20 wt% sodium ascorbyl phosphate (vitamin C);
0.025 wt% citric acid monohydrate;
1.0 wt% alexidine chloride, 0.5% solution;
0.20 wt% licorice root (glycyrrhiza glabra) extract in glycerin;
0.18 wt% cucumber fruit (cucumis sative) extract in glycerin;
0.18 wt% eyebright (euphrasia officinalis) extract in glycerin;
0.2 wt% sorbitan oleate decylglucoside crosspolymer; and
0.6 wt% decyl glucoside.

74. The formulation according to claim 34, comprising:
0.10 wt% sodium hyaluronate;
0.55 wt% propanediol;
0.12 wt% ectoine;
0.10 wt% d-panthenol (pro-vitamin B5);
0.45 wt% niacinamide (vitamin B3);
0.20 wt% sodium ascorbyl phosphate (vitamin C);
0.025 wt% citric acid monohydrate;
1.5 wt% alexidine chloride, 0.5% solution;
0.18 wt% licorice root (glycyrrhiza glabra) extract in glycerin;
0.10 wt% cucumber fruit (cucumis sative) extract in glycerin;
0.01 wt% eyebright (euphrasia officinalis) extract in glycerin;
0.3 wt% sorbitan oleate decylglucoside crosspolymer; and
0.06 wt% decyl glucoside.

75. The formulation according to claim 34, wherein the formulation is stable.

76. The formulation according to claim 34, wherein the formulation is shelf-stable.

77. The formulation according to claim 34, wherein, the pH of the formulation after storage at 20-25° C. for 24 months, is not more than 0.75 pH, such as not more than 0.6 pH units, not more than 0.5 pH units, not more than 0.4 pH units, not more than 0.3 pH units, not more than 0.2 pH units, not more than 0.1 pH units, of the pH of the formulation prior to the storage.

78. The formulation according to claim 34, wherein the osmolality of the formulation after storage at 20-25° C. for 24 months is within 40 mOsm, such as within 35 mOsm, within 30 mOsm, within 25 mOsm, within 20 mOsm, within 15 mOsm, within 10 mOsm, within 5 mOsm, or within 1 mOsm of the osmolality of the formulation prior to the storage.

79. The wipe according to claim 1, wherein the formulation is stable.

80. The wipe according to claim 1, wherein the formulation is shelf-stable.

81. The wipe according to claim 1, wherein the pH of the formulation after storage at 20-25° C. for 24 months, is not more than 0.75 pH, such as not more than 0.6 pH units, not more than 0.5 pH units, not more than 0.4 pH units, not more than 0.3 pH units, not more than 0.2 pH units, not more than 0.1 pH units, of the pH of the formulation prior to the storage.

82. The wipe according to claim 1, wherein the osmolality of the formulation after storage at 20-25° C. for 24 months is within 40 mOsm, such as within 35 mOsm, within 30 mOsm, within 25 mOsm, within 20 mOsm, within 15 mOsm, within 10 mOsm, within 5 mOsm, or within 1 mOsm of the osmolality of the formulation prior to the storage.
